(19) United States
(12) Patent Application Publication
(10) Pub. No.: US 2024/0219361 A1
(43) Pub. Date: Jul. 4, 2024

(54) A METHOD AND SYSTEM FOR ANALYZING FLUID INCLUSIONS

(71) Applicant: COMMONWEALTH SCIENTIFIC AND INDUSTRIAL RESEARCH ORGANISATION, Acton, Australian Capital Territory (AU)

(72) Inventors: Julien Francois Roger BOURDET, Acton, Australian Capital Territory (AU); Matthew JOSH, Acton, Australian Capital Territory (AU); Charles Hamilton HEATH, Acton, Australian Capital Territory (AU)

(21) Appl. No.: 18/560,657

(22) PCT Filed: May 10, 2022
(86) PCT No.: PCT/AU2022/050436

§ 371 Date: Nov. 13, 2023

(30) Foreign Application Priority Data
May 14, 2021 (AU) 2021901449

(51) Int. Cl. G01N 33/00; E21B 49/02; G01N 1/28; G01N 1/38; G01N 1/44; G01N 33/24

(52) U.S. Cl. CPC G01N 33/0021; E21B 49/02; G01N 1/286; G01N 1/38; G01N 1/44; G01N 33/0014; G01N 33/24; G01N 2001/2866

(57) ABSTRACT

A method for detecting and quantifying elemental mercury vapor in one or more solid sample. The method being particularly suitable for detecting and quantifying elemental mercury vapor in solid samples obtained from a petroleum reservoir by subjecting the solid samples to conditions that cause the release of one or more volatile compound therefrom including elemental mercury vapor, using a carrier gas to transport the released one or more volatile compound into a first trap to capture one or more volatile compound other than the elemental mercury vapor, and finally into an additional trap downstream of the first trap to capture the elemental mercury vapor for analysis and quantification.

### PRIORITY DOCUMENTS

The present application claims priority from Australian Provisional Patent Application No. 2021901449 titled “A METHOD AND SYSTEM FOR ANALYSING FLUID INCLUSIONS” and filed on 14 May 2021, the content of which is hereby incorporated by reference in its entirety.

### TECHNICAL FIELD

The present disclosure relates to a method and system for detecting elemental mercury vapour in solid samples. In a particular embodiment, the present disclosure relates to a system and method for detecting elemental mercury vapour in solid samples obtained from a petroleum reservoir to predict risk of mercury contamination.

### BACKGROUND

Mercury is a naturally occurring element that is often found in trace amounts in rock formations. In addition to health, safety and environmental issues, the presence of mercury can potentially have a large impact on the economics of drilling in these rock formations. For example, mercury is found in trace amounts in most petroleum reservoirs around the world and the presence of mercury can have a significant impact on the production of hydrocarbons from petroleum reservoirs. Although the concentration of mercury in a given petroleum reservoir may be considered low, its cumulative effect can be serious.

One of the common risks associated with the presence of mercury is that, it may form amalgams with a variety of metals, potentially causing liquid metal embrittlement (LME) and metal component corrosion of processing equipment. Accordingly, a variety of methods and technologies exist for detecting mercury content in a rock formation. Most of these existing methods and technologies require the capture or collection of data or samples during the drilling phase (such as wireline formation testing, drillstem testing, or core sample analysis). However, one of the major drawbacks of these existing methods is that there is an associated high cost, as they often require expensive tooling and can only be performed during the drilling phase (i.e. the data or samples required may only be obtained by sacrificing operational time).

Mercury can be present in a variety of forms, some forms being volatile, while others are less volatile. With respect to petroleum reservoirs, it is elemental mercury vapour which is considered a volatile form of mercury that is most detrimental to the production of hydrocarbons.

There is thus a need for a method and a system for detecting mercury contamination in petroleum reservoirs and other rock formations at a low cost and/or does not require dedicating additional time during the drilling phase. Alternatively, or in addition, there is a need for improved methods of predicting the risk of mercury contamination of a petroleum reservoir or rock formation to assist in evaluating the economics of field development, selection and design of production plant and equipment.

It is against this background and the problems and difficulties associated therewith, that the present invention has been developed.

### SUMMARY

Embodiments of the present disclosure relate to a method for detecting and quantifying elemental mercury vapour in one or more solid sample. The method being particularly suitable for detecting and quantifying elemental mercury vapour in solid samples obtained from a petroleum reservoir by subjecting the solid samples to conditions that cause the release of one or more volatile compound therefrom including elemental mercury vapour, using a carrier gas to transport the released one or more volatile compound into a first trap to capture one or more volatile compound other than the elemental mercury vapour, and finally into an additional trap downstream of the first trap to capture the elemental mercury vapour. In use, the method allows for analysis of the one or more volatile compound in the first trap to determine the composition of the one or more volatile compound, and analysis of the elemental mercury vapour in the additional trap to quantify the elemental mercury vapour released from the solid sample.

According to a first aspect, there is provided a method for detecting and quantifying elemental mercury vapour in solid samples, the method comprising: inserting one or more solid sample into an extraction chamber; introducing a carrier gas at an inlet of the extraction chamber and subjecting the one or more solid sample to conditions that cause the release of one or more volatile compound including elemental mercury vapour, whereby a loaded gas comprising the carrier gas and the one or more volatile compound, including elemental mercury vapour, is created and directed to an outlet of the extraction chamber, and analysing the loaded gas from the outlet to selectively detect and quantify elemental mercury vapour.

In one embodiment, one or more trap downstream of the outlet receives the loaded gas.

In one embodiment, the one or more trap comprises a first trap that receives the loaded gas and captures the one or more volatile compound other than elemental mercury vapour to provide a stripped gas.

In one embodiment, the one or more trap further comprises an additional trap downstream of the first trap that receives the stripped gas and captures the elemental mercury vapour.

In one embodiment, the one or more volatile compound captured by the first trap is analysed using gas chromatography or mass spectroscopy analysis to quantify and identify the one or more volatile compound released from the solid sample.

In one embodiment, the elemental mercury vapour captured by the additional trap is analysed by a mercury analyser to quantify the elemental mercury released from the solid sample.

In one embodiment, the additional trap is an amalgamation gold trap.

In one embodiment, the additional trap is analysed by a mercury analyser to quantify the elemental mercury vapour released from the solid sample.

In one embodiment, the first and additional traps are configured either in series or in parallel.

In one embodiment, the one or more volatile compound comprises volatile organic compounds (VOCs).

In one embodiment, the VOCs comprise volatile hydrocarbons.

In one embodiment, the volatile hydrocarbons comprise one or more of methane, ethane, propane, n-butane, iso-butane, 1-butene, n-pentane, iso-pentane, hexane, cyclo-pentane, 3-methylpentane, methylcyclopentane, methylcyclohexane, n-heptane, n-octane, furan, methylfuran and BTEX.

In one embodiment, BTEX comprises any one or more of hydrocarbon compounds benzene, toluene, ethylbenzene and xylene.

In one embodiment, the elemental mercury vapour and the one or more volatile compound are released from fluid inclusions in the one or more solid sample.

In one embodiment, the carrier gas comprises either Argon or Nitrogen.

In one embodiment, the stripped gas comprising the carrier gas is discharged downstream from the additional trap.

In one embodiment, the one or more solid sample is provided from different locations within a petroleum reservoir.

In one embodiment, the one or more solid sample comprises any one or more of drill cuttings or core samples from a petroleum reservoir.

In one embodiment, the conditions include any one or more of crushing, milling, agitating or heating of the one or more solid sample.

According to a second aspect, there is provided a system for detecting and quantifying elemental mercury vapour in solid samples, the system comprising: an extraction chamber comprising a receptacle, an inlet and an outlet in fluid connection with the receptacle, and a comminuting member; a gas source comprising a carrier gas and in fluid connection with the inlet; one or more trap in fluid connection with the outlet; and wherein the extraction chamber is configured to comminute the one or more solid sample to cause the release of one or more volatile compound, including elemental mercury vapour, which is directed toward the outlet by injection of the carrier gas to create a loaded gas, whereby the loaded gas is received by the one or more trap to allow analysis of the loaded gas to selectively detect and quantify elemental mercury vapour.

In one embodiment, the one or more trap comprises a first trap that receives the loaded gas and captures the one or more volatile compound other than elemental mercury vapour to provide a stripped gas.

In one embodiment, the one or more trap further comprises an additional trap downstream of the first trap that receives the stripped gas and captures the elemental mercury vapour.

In one embodiment, the elemental mercury vapour captured by the additional trap is analysed to quantify the elemental mercury vapour released from the one or more solid sample and assess the risk of mercury contamination during drilling and production operations of a petroleum reservoir.

In one embodiment, the extraction chamber is agitated to comminute the one or more solid sample via the comminuting member.

In one embodiment, the comminuting member is any one of a puck mill, a ball mill, a ring mill, a rock crusher, an anvil or a hammer element.

In one embodiment, the comminuted (or residual) one or more solid sample is weighed and compared to the elemental mercury vapour captured by the additional trap for calculation of fluid inclusion content of the solid sample.

According to a further aspect, there is provided a method for detecting and quantifying elemental mercury vapour in solid samples, the method comprising:
inserting one or more solid sample into an extraction chamber;
introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury vapour, is created and directed to an outlet of the extraction chamber;
receiving the loaded gas at a first trap downstream of the outlet to capture the one or more volatile compound, other than elemental mercury vapour, to provide a stripped gas; and
receiving the stripped gas at an additional trap downstream of the first trap to selectively detect and, or capture elemental mercury vapour within the stripped gas for subsequent analysis and quantification.

According to a further aspect, there is provided a method of predicting risk of mercury contamination of a petroleum reservoir by detecting elemental mercury vapour in solid samples, the method comprising: providing one or more solid sample from locations within the petroleum reservoir; inserting the one or more solid sample into an extraction chamber; introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury, is created and directed to an outlet of the extraction chamber; receiving the loaded gas at one or more trap downstream of the outlet to selectively detect elemental mercury vapour within the loaded gas; and analysing the elemental mercury vapour detected to assess the risk of mercury contamination of the petroleum reservoir.

According to yet a further aspect, there is provided a method of predicting and assessing risk of mercury contamination of a petroleum reservoir, the method comprising: (a) providing one or more solid sample from a location within the petroleum reservoir; (b) inserting the one or more solid sample into an extraction chamber; (c) introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury vapour, is created and directed to an outlet of the extraction chamber; (d) receiving the loaded gas at a first trap downstream of the outlet to capture the one or more volatile compound, other than elemental mercury vapour, to provide a stripped gas; (e) subsequently receiving the stripped gas at an additional trap downstream of the first trap to capture the elemental mercury vapour, wherein the elemental mercury vapour captured by the additional trap is analysed to quantify the elemental mercury vapour released from the solid sample; (f) and repeating steps (a) to (e) for one or more subsequent solid sample from alternate locations within the petroleum reservoir, wherein analysed and quantified elemental mercury vapour of the one or more solid sample provided from various locations within the petroleum reservoir are used to predict and assess the risk of mercury contamination of the petroleum reservoir during drilling and production operations.

### BRIEF DESCRIPTION OF THE FIGURES

Embodiments of the present disclosure will be discussed with reference to the accompanying figures wherein:

FIG. 1 is a schematic illustrating a petroleum reservoir, a drilled well and solid samples obtained from various locations (depths) within the reservoir;

FIG. 2 is a schematic process diagram illustrating a system for detecting elemental mercury vapour in solid samples;

FIG. 3 is a perspective view of an extraction chamber for comminuting solid samples obtained from a petroleum reservoir;

FIG. 4 is a top view of the extraction chamber of FIG. 3;

FIG. 5 is a sectional view of the extraction chamber taken along line A-A of FIG. 4;

FIG. 6 is a process flow diagram illustrating a method for determining hydrocarbon yield and mercury contamination of a petroleum reservoir using solid samples from said petroleum reservoir; and

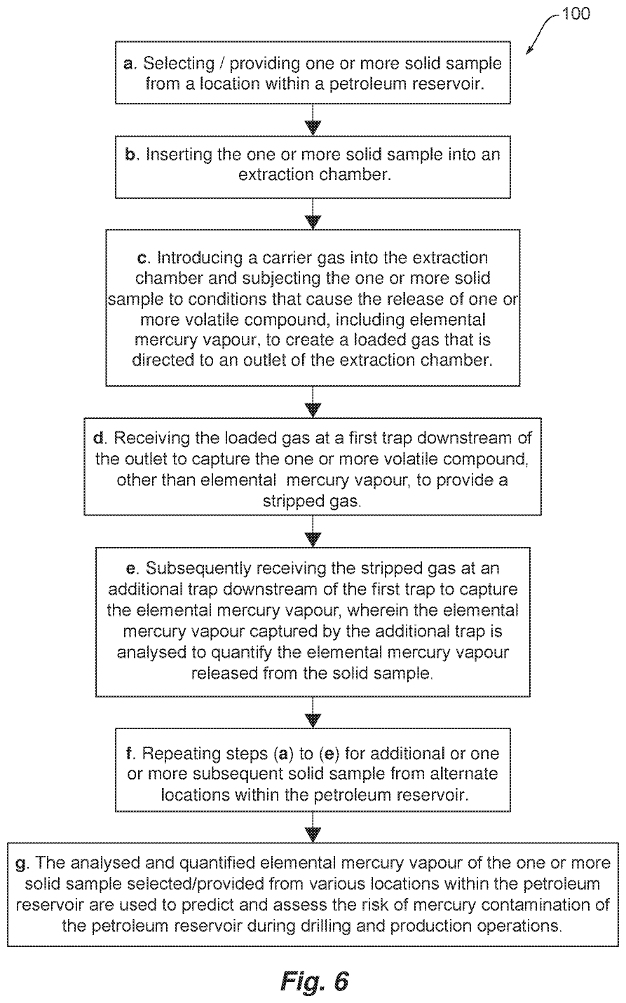

FIG. 7 is a schematic process diagram illustrating an alternate system to that in FIG. 2, wherein the system comprises an intermediary trap.

In the following description, like reference characters designate like or corresponding parts throughout the figures.

### DESCRIPTION OF EMBODIMENTS

Referring to any one of the Figures, there is disclosed both a method (100) and system (200) for detecting and quantifying elemental mercury vapour (also referred to as “Hg⁰”, or elemental mercury in a gaseous phase) from fluid inclusions in a solid sample (300) obtained from a rock formation, such as a petroleum reservoir (400). Subsequently, the method (100) and system (200) involves analysis and quantification of elemental mercury vapour which may be utilised to predict risk of mercury contamination of the petroleum reservoir (400). In addition to elemental mercury vapour, other one or more volatile compound is also typically released from the fluid inclusions of the solid sample (300). The one or more volatile compound may be separated and independently analysed and quantified to determine hydrocarbon yield of the petroleum reservoir (400).

Elemental mercury vapour is contained or enclosed within fluid inclusions of solid samples (300); fluid inclusions are micron-scale sized chambers in minerals (such as quartz, feldspar, carbonate, pyrite etc.). Elemental mercury vapour is known, to those skilled in the art, as a ‘highly volatile form of mercury’ and is in a gaseous or vapour form that is contained or enclosed within the fluid inclusions of solid samples (300). That is to say, those solid samples (300) at atmospheric conditions are capable of containing or enclosing, within their fluid inclusions, elemental mercury vapour. The method (100) and system (200) of this present disclosure subjects the solid samples (300) to conditions that result or cause the release of elemental mercury vapour (and other volatile compounds) from fluid inclusions.

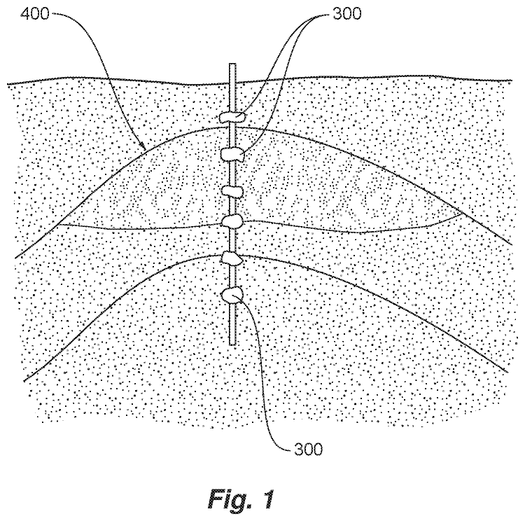

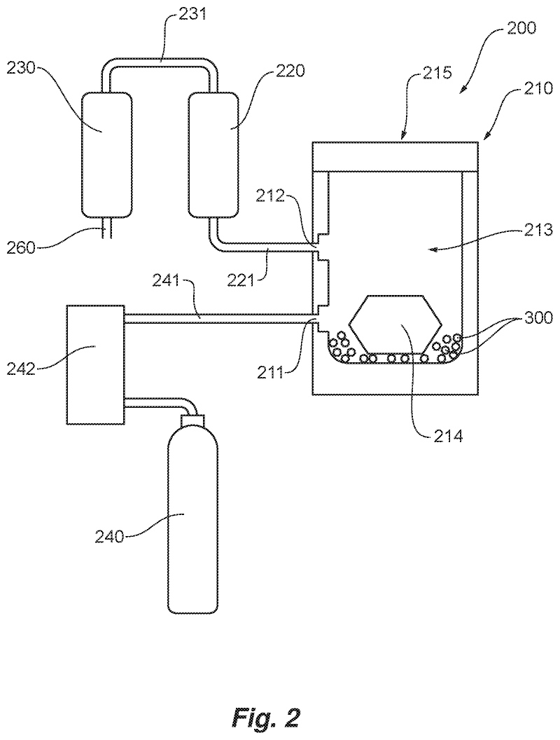

Referring particularly to FIGS. 1 and 2, there is illustrated an embodiment of the method (100) for detecting and quantifying elemental mercury vapour in solid samples (300). The method (100) comprising:
(a) inserting one or more solid sample (300) into an extraction chamber (210);
(b) introducing a carrier gas at an inlet (211) of the extraction chamber (210) and subjecting the one or more solid sample (300) to conditions that cause the release of one or more volatile compound, including elemental mercury vapour, whereby a loaded gas comprising the carrier gas and the one or more volatile compound, including elemental mercury vapour, is created and directed to an outlet (212) of the extraction chamber (210); and
(c) analysing the loaded gas from the outlet (212) to selectively detect and quantify elemental mercury vapour.

The one or more solid sample (300) required at step (a) may be obtained from the petroleum reservoir (400), in order to predict the distribution of mercury within the petroleum reservoir (400) and its surrounding geographical area. The one or more solid sample (300) may comprise any one or more of drill cuttings, core samples or other rock samples obtained from the petroleum reservoir (400), via known methods systematically performed during drilling of the petroleum reservoir (400). Referring particularly to FIG. 1, it will be appreciated that in order to best predict the distribution of mercury within the reservoir (400), the one or more solid sample (300) will typically be provided from different locations within the reservoir (400). This Figure particularly illustrates that the one or more solid sample (300) may be obtained as a number of successive samples at different depths, in different compartments and/or in different sand units in the petroleum reservoir (400) to permit the best prediction of mercury distribution. In an alternative to this Figure, the one or more solid sample (300) may be obtained from a number of different wells drilled in the same petroleum reservoir (400) to allow for prediction of mercury distribution.

The one or more solid sample (300) comprises fluid inclusions from which elemental mercury vapour and other volatile compounds must be released from, in order to be subsequently analysed and quantified. Any suitable conditions can be used to release the one or more volatile compound and elemental mercury vapour from the one or more solid sample (300). For example, the conditions that the extraction chamber (210) subjects the one or more solid sample (300) to, may include one or more of: physical break down or comminution of the one or more solid sample (300) such as by any one or more of crushing, milling, slicing, cutting; agitating the one or more solid sample (300) to release one or more volatile compound and elemental mercury vapour; heating the one or more solid sample (300) to release one or more volatile compound and elemental mercury vapour; exposing the one or more solid sample (300) to a vacuum in the extraction chamber (210) to release one or more volatile compound and elemental mercury vapour; or exposing the one or more solid sample (300) to a positive pressure in the extraction chamber (210) to release one or more volatile compound and elemental mercury vapour. Any one or more of these conditions result in the release of elemental mercury vapour and other volatile compounds from the one or more solid sample (300).

The one or more volatile compound released may comprise volatile organic compounds (otherwise known as VOCs). The VOCs may be volatile hydrocarbons such as one or more of methane, ethane, propane, n-butane, iso-butane, 1-butene, n-pentane, iso-pentane, hexane, cyclo-pentane, 3-methylpentane, methylcyclopentane, methylcyclohexane, n-heptane, n-octane, furan, methylfuran and BTEX (otherwise known as any one or more of hydrocarbon compounds benzene, toluene, ethylbenzene and xylene). It will be appreciated that as the one or more solid sample (300) is obtained from the petroleum reservoir (400), the other one or more volatile compound may also comprise H₂S, H₂O, CO₂, N₂ and other fluids commonly occurring within fluid inclusions of samples obtained from petroleum reservoirs. The VOCs are often referred to as ‘volatile’ due to their composition being mostly of small molecules that typically volatilise at atmospheric conditions. When contained or enclosed within the fluid inclusions of the one or more solid sample (300), the VOCs may be in a solid, liquid or gaseous form. However once released (via any one of the suitable conditions disclosed above), the VOCs are typically in a gaseous form as they are volatilised.

The present inventors have surprisingly found that it may be particularly advantageous to remove VOCs from the loaded gas prior to trapping elemental mercury vapour as these VOCs can interfere with elemental mercury capture and trapping. By prior removal of VOCs, the potential for one or more of the VOCs to be falsely detected and/or quantified as elemental mercury vapour is mitigated. Atomic fluorescence spectroscopy is a commonly utilised mercury analysis method utilised in the petroleum industry, where a sample is typically converted into gaseous atoms and molecules such that any contained mercury is excited to a high electronic energy level by a light source, and subsequently the atoms are deactivated by the emission of a photon, this emission is measured fluorescence that represents mercury. The present inventors through experimentation noted that in mercury analysers, such as atomic fluorescence spectroscopy, VOCs could also be excited by the same light source and would also emit a fluorescence resulting in a false positive of mercury detection. Thus, the trapping or removal of VOCs first provides for a more accurate representation of the presence of elemental mercury vapour in a solid sample (300).

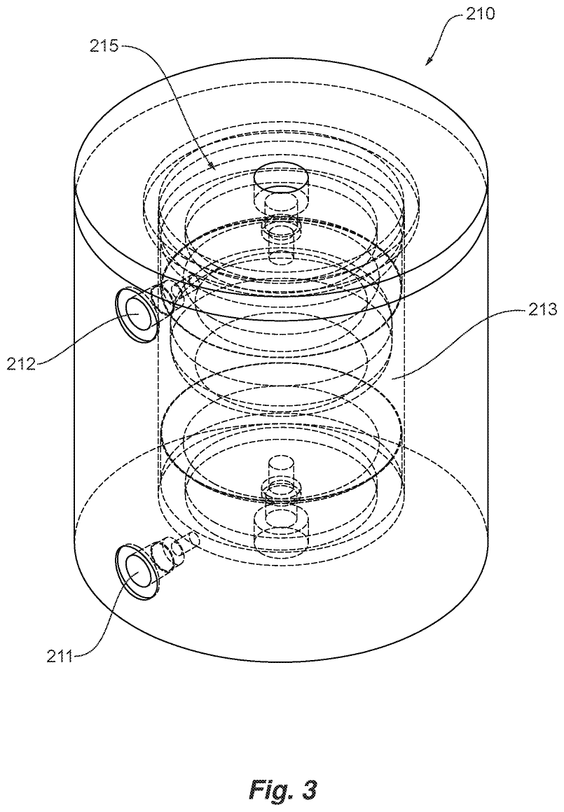

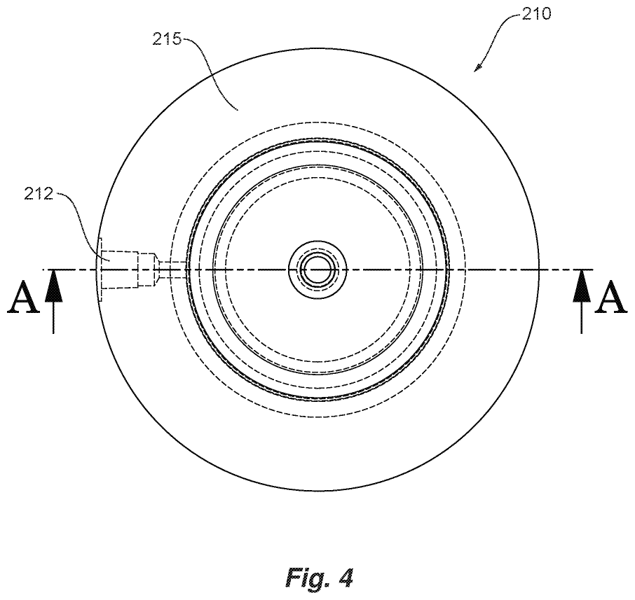

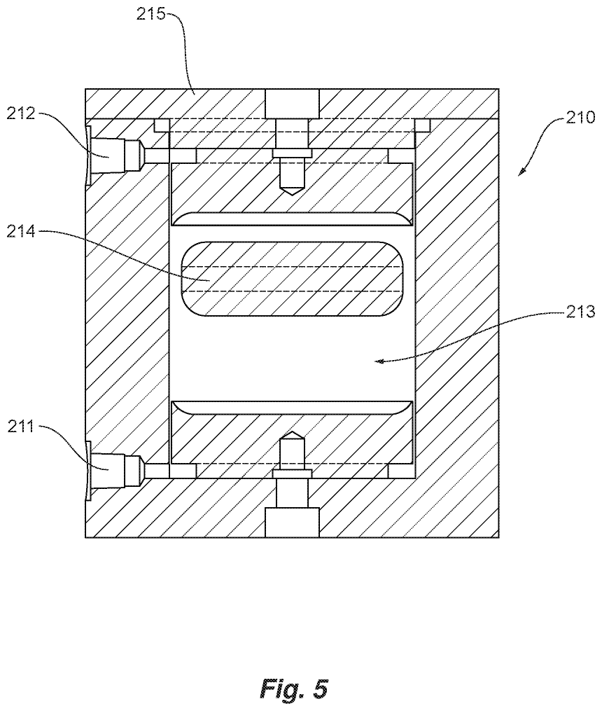

In one embodiment, referring now to any one of FIGS. 2 to 5, the extraction chamber (210) utilised at step (a), may also comprise a receptacle (213) sized and shaped to receive the one or more solid sample (300) therein, and a comminuting member (214). The comminuting member (214) may be disposed within the receptacle (213), wherein the receptacle (213) still comprises space to receive the one or more solid sample (300) therein. In this way, with the one or more solid sample (300) within the receptacle (213) with the comminuting member (214), the extraction chamber (210) may be agitated to comminute the one or more solid sample (300) via the comminuting member (214).

The receptacle (213) may be constructed of a sufficiently solid material, for example steel, such that the receptacle (213) is able to withstand the agitation of the extraction chamber (210) to comminute the one or more solid sample (300) via the comminuting member (214) without discharging the contents of the receptacle (213). Accordingly, the receptacle (213), in one embodiment, may be a container sized and shaped to receive the one or more solid sample (300) and the comminuting member (214) therein. In the Figures, the receptacle (213) is illustrated as a cylindrical shape, however it will be appreciated that the receptacle (213) may take other shapes, such that it is able to receive the one or more solid sample (300) and the comminuting member (214) therein, and be able to withstand the agitation of the extraction chamber (210) to comminute the one or more solid sample (300).

In this embodiment, the comminuting member (214) may be any one of a puck mill, a ball mill, a ring mill, a rock crusher, an anvil or a hammer element. Whereby the released elemental mercury vapour and other one or more volatile compound from the one or more solid sample (300) is contained within the receptacle (213), until subsequently at step (b) the carrier gas is injected at the inlet (211) to create the loaded gas comprising the carrier gas, the one or more volatile compound, and the elemental mercury vapour, directing the loaded gas from the receptacle (213) to the outlet (212) of the extraction chamber (210). It will be appreciated that the inlet (211), the outlet (212) and the receptacle (213) therebetween are in fluid communication, and that the extraction chamber (210) is sufficiently sealed to contain the released elemental mercury vapour, one or more volatile compound and the subsequently created loaded gas. The comminuting member (214) is particularly designed so as to permit the carrier gas, the one or more volatile compound, the elemental mercury vapour and the created loaded gas, to be transported through an internal volume of the receptacle (213) as the extraction chamber (210) is agitated to comminute the one or more solid sample (300).

In this embodiment, it will be appreciated that the one or more solid sample (300) may be subjected to the conditions imposed by the extraction chamber (210) for a sufficient amount of time to completely release the one or more volatile compound and the elemental mercury vapour from the fluid inclusions. It is estimated that for a 3 gram solid sample (300), it will typically take approximately 2 minutes of subjecting said sample (300) to the conditions within the extraction chamber (210), whilst injecting the carrier gas required by step (b), to completely release all of the one or more volatile compound and the elemental mercury vapour from the fluid inclusions and capture these to create the loaded gas.

In this embodiment, the extraction chamber (210) may further comprise a removable lid (215), whereby the lid (215) comprises a seal to create a gas tight seal to contain the released elemental mercury vapour, one or more volatile compound and the subsequently created loaded gas within the receptacle (213). The lid (215) is removable, so as to allow the one or more solid sample (300) to be placed within the receptacle (213) of the extraction chamber (210), and to allow the comminuting member (214) to be removed or replaced within the receptacle (213). It will be appreciated that the comminuting member (214) may be removed or replaced due to being worn, or based on the conditions to subject the one or more solid sample (300) to, a particular comminuting member (214) may be selected to achieve releasing the elemental mercury vapour and one or more volatile compound from the fluid inclusions of the one or more solid sample (300).

In this embodiment, an additional step (a) (i), subsequent to step (a) and prior to (b), may be carried out whereby the carrier gas is injected from the inlet (211), into the receptacle (213) and out the outlet (212) prior to comminuting the one or more solid sample (300). In this way, the internal volume of the receptacle (213), the inlet (211) and the outlet (212) are essentially flushed with the carrier gas prior to the release of elemental mercury vapour and the other volatile compounds from the fluid inclusions of the one or more solid sample (300), to rid the extraction chamber (210) from any contaminants.

In any one of the above embodiments, it will be appreciated that the carrier gas may be injected from the inlet (211) into the receptacle (213) prior to, during or after comminuting (or subjecting to conditions) the one or more solid sample (300). In one example, the carrier gas may be injected from the inlet (211) into the receptacle (213) during comminuting of the one or more solid sample (300) (or subjecting the one or more solid sample (300) to conditions). In this example, the carrier gas is injected whilst one or more volatile compound, including elemental mercury vapour, is released from the one or more solid sample (300), creating the loaded gas comprising the carrier gas and the one or more volatile compound, including elemental mercury vapour, and directing the created loaded gas to the outlet (212). In another example, the carrier gas may be injected from the inlet (211) into the receptacle (213) after comminuting the one or more solid sample (300). In this example, the one or more solid sample (300) is comminuted (or subjected to conditions) that cause the release of one or more volatile compound, including elemental mercury vapour, within the receptacle (213) of the extraction chamber (210). The released one or more volatile compound, including elemental mercury vapour, may be held sealed within the extraction chamber (210) for a period of time, then subsequently injecting the carrier gas from the inlet (211) into the receptacle (213) to create and direct the loaded gas comprising the carrier gas and the one or more volatile compound, including elemental mercury vapour, to the outlet (212). Thus, it will be appreciated that the method (100) may incorporate injecting the carrier gas at a time prior to, during or after subjecting the one or more solid sample (300) to conditions that cause the release of one or more volatile compound, including elemental mercury vapour.

In one embodiment, the carrier gas (or transport gas) is an inert gas that is selected to be non-reactive with either the elemental mercury vapour or the one or more volatile compound. In this embodiment, the carrier gas may comprise, but is not limited to, either Argon or Nitrogen for example. Also in this embodiment, it is possible for the carrier gas to be “air” that has been analysed and/or specifically prepared, such that it is non-reactive with and does not contain either the elemental mercury vapour or the one or more volatile compound. It will be appreciated that the use of “air”, without analysis and/or preparation, as the carrier gas, is not desirable as “air” typically comprises both oxygen and water vapour that may have some reactive properties with either the elemental mercury vapour or the one or more volatile compound. Additionally, the use of “air” (without analysis and/or preparation) as the carrier gas, is disadvantageous as it is possible that it contains trace amounts of mercury. Thus, it is advantageous for the carrier gas to be an inert gas that is mercury free.

In one embodiment, referring particularly to FIG. 2 and the method at step (b), the carrier gas may be injected into the inlet (211) of the extraction chamber (210) from a gas source (240) located upstream from the inlet (211). The gas source (240) may be a gas bottle or another gas source containing the carrier gas, able to inject the carrier gas into the extraction chamber (210), at a controlled injection or flow rate, via an injection line (241). In this embodiment, a gas flow meter (242) may be utilised between the gas source (240), the injection line (241) and the inlet (211) of the extraction chamber (210). The gas flow meter (242) is in fluid connection with the gas source (240), and the injection line (241) connects the gas flow meter (242) to the inlet (211) of the extraction chamber (210). In use, the gas flow meter (242) may be used to assist in monitoring volume and pressure of the carrier gas introduced (or injected) into the extraction chamber (210). By way of example, where the carrier gas is inert gas Argon, it is estimated that the ideal pressure and flow rate to inject Argon into the extraction chamber (210) is 9 psi measured at the gas flow meter (242), which equates to 500 mL/min flow rate to completely capture elemental mercury vapour and one or more volatile compound released from fluid inclusions of the one or more solid sample (300) to create the loaded gas, and direct the created loaded gas out the outlet (212) of the extraction chamber (210).

Referring still to FIG. 2 and the method at step (c), in one embodiment, the loaded gas is ejected out the outlet (212) of the extraction chamber (210), and is analysed so as to selectively detect the presence of elemental mercury vapour. As illustrated, the outlet (212) may be connected to one or more trap (220, 230), whereby the one or more trap (220, 230) is downstream of the outlet (212) and receives the loaded gas therefrom. The one or more trap (220, 230) may be connected to the outlet (212) via a first ejection line (221). In this embodiment, the one or more trap (220, 230) may comprise a first trap (220) that receives the loaded gas and captures the one or more volatile compound (other than elemental mercury vapour) to provide a stripped gas. Also in this embodiment, the first trap (220) is selected based on its ability to capture the one or more volatile compound from the loaded gas being received therein, to create the stripped gas which comprises the carrier gas and the elemental mercury vapour. The first trap (220) may be a device such as an Tenax-TA Trap known for its ability to capture and remove one or more volatile compound, such as volatile organic compounds (VOCs), where the VOCs may be volatile hydrocarbons comprising BTEX, from a flue gas (i.e. the loaded gas in this embodiment). It will be appreciated that at a high level the first trap (220) has two purposes, being to capture the one or more volatile compound for analysis and quantification such that the one or more volatile compound may be identified, and that the resultant stripped gas ejected from the first trap (220) only comprises elemental mercury vapour and the carrier gas. It will also be appreciated that the first trap (220) acts so as to ensure there are no volatile compounds in the resultant stripped gas to interfere with the subsequent detection and quantification of elemental mercury vapour.

In the above embodiments, following the completion of the method (100) steps, the one or more volatile compound captured by the first trap (220) is analysed using gas chromatography or mass spectroscopy analysis to quantify and identify the one or more volatile compound released from the one or more solid sample (300). It will be appreciated that prior to using gas chromatography or mass spectroscopy analysis (also often referred to as GC/MS or GCMS), the first trap (220) may be analysed via thermal desorption if required. Advantageously, analysis, identification and quantification of the one or more volatile compound assist in determining a hydrocarbon yield of the petroleum reservoir (400). It will be understood in determining the hydrocarbon yield of the petroleum reservoir (400), the method (100) starting from step (a) through to (c) may be repeated for a number of solid samples (300) obtained at different depths, in different compartments, in different wells and/or different sand units in the petroleum reservoir (400), such that the one or more volatile compound captured in each resultant first trap (220) are analysed to permit the best prediction of hydrocarbon yield of the petroleum reservoir (400). It will be appreciated that other analysis and quantification methods (other than GCMS) may be used to identify the one or more volatile compound captured by the first trap (220) beyond those discussed herein. Examples of other analysis and quantification methods (other than GCMS) may include; infrared spectrophotometry, ultraviolet fluorescence spectrophotometry or Raman spectroscopy.

Referring still to FIG. 2, in one embodiment, the one or more trap (220, 230) may further comprise an additional trap (230) downstream of the first trap (220) that receives the stripped gas and captures the elemental mercury vapour. As illustrated, the first trap (220) may be connected to the additional trap (230) via a second ejection line (231). In this embodiment, the additional trap (230) is selected based on its ability to capture the elemental mercury vapour from the stripped gas being received therein, to subsequently result in the stripped gas comprising only the carrier gas. The additional trap (230) may be an amalgamation gold trap comprising a gold impregnated silica, such as an AMASIL® amalgamation gold trap known for its ability to capture elemental mercury vapour from a flue gas (i.e. the stripped gas in this embodiment). It will be appreciated that it is undesirable for the one or more volatile compound (such as the VOCs, where the VOCs may be volatile hydrocarbons that comprise BTEX) to reach the additional trap (230), thus accordingly, the first trap (220) is upstream of the additional trap (230) to capture and remove the one or more volatile compound from the loaded gas to allow the stripped gas comprising the elemental mercury vapour into the additional trap (230).

In the above embodiment, following the completion of the method (100) steps, the elemental mercury vapour captured by the additional trap (230) is analysed to quantify the elemental mercury vapour released from the one or more solid sample (300), and to assess the risk of mercury contamination during drilling and production operations of the petroleum reservoir (400). The analysis of the elemental mercury vapour captured by the additional trap (230) may be via a mercury analyser (not shown) to quantify the elemental mercury vapour released from the one or more solid sample (300). The mercury analyser to analyse and quantify the mercury vapour captured by the additional trap (230) may be a commercial atomic fluorescence mercury analyser, such as a model Sir Galahad II from PSAnalytical. This type of mercury analyser provides a measurement of elemental mercury vapour in nanograms (ng). It will be understood that in assessing or predicting the risk of mercury contamination of the petroleum reservoir (400), the method (100) starting from step (a) through to (c) may be repeated for a number of solid samples (300) obtained at different depths, in different compartments, in different wells and/or different sand units in the petroleum reservoir (400), such that elemental mercury vapour captured by each resultant additional trap (230) is analysed to quantify the elemental mercury vapour released from each solid sample (300) to best assess or predict the risk of mercury contamination of the petroleum reservoir (400). It will be appreciated that other types of analysis and quantification methods may be used to analyse and quantify elemental mercury vapour captured by the additional trap (230) beyond those discussed herein.

In any one of the above embodiments, subsequent to step (b) of the method (100), the carrier gas may be continued to be injected or flowed through the extraction chamber (210), via the gas source (240) through the injection line (241) connected to the inlet (211), subsequent to the one or more solid sample (300) being completely comminuted (or subjected to the conditions to produce the one or more residual solid sample), to ensure that all of the released one or more volatile compound and elemental mercury vapour, are combined with the carrier gas to form the loaded gas.

In any one of the above embodiments, as best illustrated by FIG. 2, the one or more trap (220, 230) may be configured either in series or in parallel. That is to say, the first (220) and additional (230) trap may be configured either in series or parallel downstream of the outlet (212) of the extraction chamber (210). In this way, in one embodiment wherein the traps (220, 230) are in series, the loaded gas ejected from the outlet (212) of the extraction chamber (210) flows through the first ejection line (221) into the first trap (220), and subsequently the stripped gas comprising elemental mercury vapour is ejected from the first trap (220) and flows through the second ejection line (231) into the additional trap (230). Subsequently, once the additional trap (230) has captured the elemental mercury vapour from the stripped gas, the stripped gas comprising only the carrier gas is discharged downstream of the additional trap (230) or re-captured at a gas storage unit (not shown). In an alternative embodiment (not shown), wherein the one or more trap (220, 230) is in parallel, the loaded gas ejected from the outlet (212) of the extraction chamber (210) flows through the first extraction line (221) into the one or more trap (220, 230), and subsequently the stripped gas comprising the carrier gas is ejected from the one or more trap (220, 230) via a final ejection line (260).

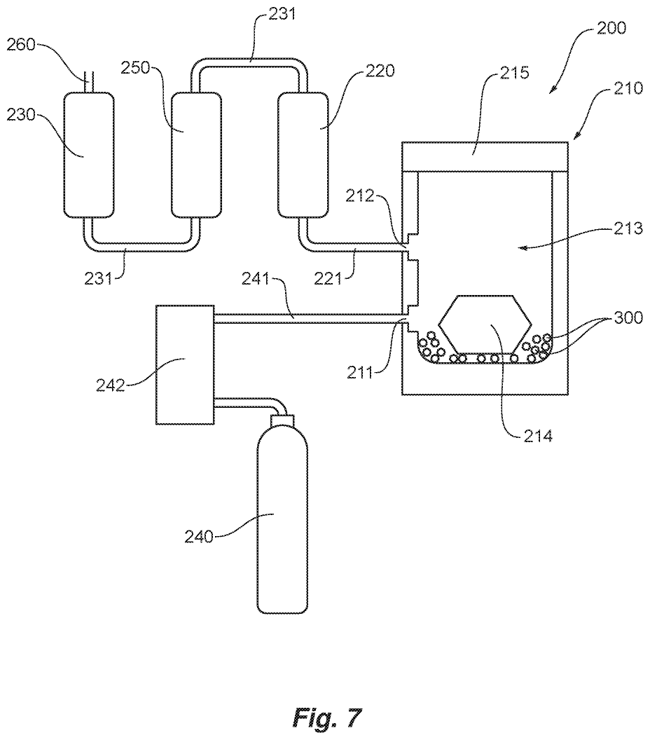

In an alternative embodiment, as illustrated by FIG. 7, at step (b) of the method (100) hydrogen sulphide (H₂S), which is a volatile non-hydrocarbon compound, may also be released from the one or more solid sample (300) while being subjected to conditions within the extraction chamber (210). That is to say, in this alternative embodiment, subjecting the one or more solid sample (300) to conditions within the extraction chamber (210) may cause the release of one or more volatile compound, including elemental mercury vapour, and H₂S. H₂S released from the one or more solid sample (300) may be in a gaseous form, and at step (b) of the method (100), the introduced carrier gas creates the loaded gas comprising the carrier gas and the one or more volatile compound, including elemental mercury vapour and H₂S. In this alternative embodiment, the one or more trap (220, 230) may further comprise an intermediary trap (250) positioned upstream of the additional trap (230). In this way, the first (220) and intermediary traps (250) are both upstream of the additional trap (230), such that the first trap (220) receives the loaded gas to capture the one or more volatile compound, other than elemental mercury vapour and H₂S, to provide a stripped gas comprising elemental mercury vapour, H₂S and the carrier gas. The intermediary trap (250) then receives the stripped gas and captures the H₂S to provide a stripped gas comprising elemental mercury vapour and the carrier gas. Subsequently the additional trap (230) receives the stripped gas comprising elemental mercury vapour and the carrier gas to capture the elemental mercury vapour. It will be appreciated that in this alternative embodiment, it is undesirable for H₂S to reach the additional trap (230), thus accordingly, the first trap (220) and the intermediary trap (250) are both located upstream of the additional trap (230). It will also be appreciated that the order in which the first (220) and intermediary trap (250) are downstream of the outlet (212) of the extraction chamber (210) is not essential; either may be directly downstream of the outlet (212) to receive the loaded gas.

Referring to either one of FIG. 2 or 7, it will be appreciated that in addition to the method (100), there is also disclosed the system (200) for detecting elemental mercury vapour in one or more solid sample (300). The system (200) comprises the extraction chamber (210) comprising the receptacle (213), the inlet (211) and the outlet (212) in fluid connection with the receptacle (213), and the comminuting member (214). The system also comprising the gas source (240) comprises the carrier gas, the gas source (240) being in fluid connection with the inlet (211) of the extraction chamber (210) to introduce the carrier gas therein. The one or more trap (220, 230) being in fluid connection with the outlet (212) of the extraction chamber (210) via the first ejection line (221). Wherein, in use, the extraction chamber (210) is configured to comminute the one or more solid sample (300) to cause the release of one or more volatile compound, including elemental mercury vapour, which is directed toward the outlet (212) by injection of the carrier gas to create the loaded gas, whereby the loaded gas is received by the one or more trap (220, 230) to allow analysis of the loaded gas to selectively detect elemental mercury vapour. It will be appreciated that like features of this system (200) to those features of the method (100) are analogous, and the embodiments above which describe additional function or form of these features apply to the system (200) and will not be repeated.

In any one of the above embodiments, the comminuted (or residual) one or more solid sample (300) is removed from the receptacle (213) of the extraction chamber (210) and weighed, to compare the weight of the comminuted (or residual) one or more solid sample (300) to the elemental mercury vapour captured by the additional trap (230) for calculation of fluid inclusion content of the one or more solid sample (300). This calculation may be used to ascertain the fluid inclusion content of not only the one or more solid sample (300), but when compared to a number of solid samples (300) subjected to the method (100) or the system (200), the fluid inclusion content of the petroleum reservoir (400) from which the one or more solid sample (300) was obtained.

In any one of the above embodiments, once the method (100) or the system (200) has been utilised for a solid sample (300) from one depth or compartment of the petroleum reservoir (400), the lid (215) of the extraction chamber (210) is removed and the comminuted (or residual) one or more solid sample (300) is removed (after being weighed) from the receptacle (213). The extraction chamber (210) and its components, including the lines (221, 231 and 241), are then all cleaned and flushed with compressed air in preparation of the next solid sample (300) from another depth or compartment of the petroleum reservoir (400) for analysis. Subsequently, before the insertion of the next solid sample (300), the extraction chamber (210) and its components including the lines (221, 231 and 241) may be cleaned and flushed with an inert gas that is non-reactive with either the elemental mercury vapour or the one or more volatile compound (such as Argon or Nitrogen). It will be understood that the first (220) and additional (230) trap (also intermediate trap (250), if any) are typically replaced with new traps (220, 230, 250) for one or more subsequent solid sample (300).

In any one of the above embodiments, the extraction chamber (210) is sufficiently sealed or enclosed. It will be appreciated that in this way, the extraction chamber (210) is able to fully contain the released elemental mercury vapour, H₂S (if any), and other one or more volatile compound from the one or more solid sample (300), until the carrier gas is injected at the inlet (211) to create the loaded gas and subsequently exit the extraction chamber (210) via the outlet (212). Advantageously, all of the elemental mercury vapour released from the one or more solid sample (300) is subsequently captured for quantification and analysis.

A preferred embodiment of the method (100) for detecting and quantifying elemental mercury vapour in solid samples (300) may comprise the following steps:
(a) inserting one or more solid sample (300) into an extraction chamber (210);
(b) introducing a carrier gas at an inlet (211) of the extraction chamber (210) and subjecting the one or more solid sample (300) to conditions that cause the release of one or more volatile compound, including elemental mercury vapour, whereby a loaded gas comprising the carrier gas and the one or more volatile compound, including elemental mercury vapour, is created and directed to an outlet (212) of the extraction chamber (210);
(c) receiving the loaded gas at a first trap (220) downstream of the outlet (212) to capture the one or more volatile compound, other than elemental mercury vapour, to provide a stripped gas; and
(d) receiving the stripped gas at an additional trap (230) downstream of the first trap (220) to selectively detect and/or capture elemental mercury vapour within the stripped gas for subsequent analysis and quantification.

It will be appreciated that in addition to the method (100) and the system (200), utilising the above embodiments, there is also disclosed a method of predicting risk of mercury contamination of a petroleum reservoir (400) by detecting elemental mercury vapour in one or more solid sample (300). This method comprising:
(a) providing one or more solid sample (300) from locations within a petroleum reservoir (400);
(b) inserting the one or more solid sample (300) into an extraction chamber (210);
(c) introducing a carrier gas at an inlet (211) of the extraction chamber (210) and subjecting the one or more solid sample (300) to conditions that cause the release of one or more volatile compound, including elemental mercury vapour, whereby a loaded gas comprising the carrier gas and the one or more volatile compound, including elemental mercury vapour, is created and directed to an outlet (212) of the extraction chamber (210);
(d) receiving the loaded gas at one or more trap (220, 230) downstream of the outlet (212) to selectively detect elemental mercury vapour within the loaded gas; and
(e) analysing and quantifying the elemental mercury vapour detected to assess the risk of mercury contamination of the petroleum reservoir (400).

It will further be appreciated, once more utilising the above embodiments, that there is also disclosed a method of predicting and assessing risk of mercury contamination of a petroleum reservoir (400). This method comprising:
(a) providing one or more solid sample (300) from a location within the petroleum reservoir (400);
(b) inserting the one or more solid sample (300) into an extraction chamber (210);
(c) introducing a carrier gas at an inlet (211) of the extraction chamber (210) and subjecting the one or more solid sample (300) to conditions that cause the release of one or more volatile compound, including elemental mercury vapour, whereby a loaded gas comprising the carrier gas and the one or more volatile compound, including elemental mercury vapour, is created and directed to an outlet (212) of the extraction chamber (210);
(d) receiving the loaded gas at a first trap (220) downstream of the outlet (212) to capture the one or more volatile compound, other than elemental mercury vapour, to provide a stripped gas;
(e) subsequently receiving the stripped gas at an additional trap (230) downstream of the first trap (220) to capture the elemental mercury vapour, wherein the elemental mercury vapour captured by the additional trap (230) is analysed to quantify the elemental mercury vapour released from the one or more solid sample (300); and
(f) repeating steps (a) to (e) for one or more subsequent solid sample (300) from alternate locations within the petroleum reservoir (400), wherein analysed and quantified elemental mercury vapour of the one or more solid sample (300) provided from various locations within the petroleum reservoir (400) are used to predict and assess the risk of mercury contamination of the petroleum reservoir (400) during drilling and production operations.

Advantageously, the one or more solid sample (300) obtained from the petroleum reservoir (400) may be analysed offline and at any time subsequent to drilling of the petroleum reservoir (400) by any one of the above embodiments of the method (100) and system (200) disclosed herein. This is due to the fact that the one or more solid sample (300) may comprise rock samples or drill cuttings obtained systematically during drilling the petroleum reservoir (400), without being an additional step taking up operational time or impeding the drilling process. The rock samples or drill cuttings that make up the one or more solid sample (300) may be transported and stored for extended periods of time prior to analysis using any one of the above embodiments.

An additional advantage of any one of the methods (100) or system (200) of any one of the above embodiments, is that it is substantially lower in cost and the technologies used are more readily obtainable when compared to existing methods and technologies for detecting mercury content in petroleum reservoirs (such as wireline formation testing, drillstem testing, or core sample analysis).

It will be understood that by detecting elemental mercury vapour and volatile hydrocarbons within fluid inclusions of a sequence of solid samples obtained from petroleum reservoirs, provides information about the relationship between elemental mercury distribution and charge of the petroleum reservoir. This in turn advantageously assists in predicting the risk that the surrounding geographical area to the petroleum reservoir has of releasing mercury during hydrocarbon production.

It will also be understood that the above embodiments describing the methods and systems provide low cost methods and systems for detecting mercury contamination in a petroleum reservoir without the need for expensive tooling, and the ability to be performed offline without impeding the drilling phase. The ability to analyse and quantify mercury contamination with the methods and systems disclosed advantageously allow economic assessment of petroleum reservoirs when developing drilling and production programs. Having identified the potential risk in mercury contamination of the reservoir, mitigation plans may be implemented in drilling and production programs to minimise the risk of LME and metal component corrosion of processing equipment, while alleviating health, safety and environmental concerns.

It will further be understood that any one of the above embodiments of the method (100) or system (200) are particularly concerned with selectively detecting, quantifying and analysing elemental mercury vapour present in (contained or enclosed in) fluid inclusions of one or more solid sample (300). Elemental mercury vapour in fluid inclusions of one or more solid sample (300) remains in situ. The present inventors acknowledge that there is a variety of forms of mercury, however, forms other than elemental mercury vapour are not of concern to the present disclosure. The present inventors wish to note that in rock formations of petroleum reservoirs (400), liquid metal mercury is typically not necessarily present, and other trace forms of mercury that may be present (other than elemental mercury vapour), are not objectively detected and/or quantified by the present method (100) or system (200).

The reference to any prior art in this specification is not, and should not be taken as, an acknowledgement of any form of suggestion that such prior art forms part of the common general knowledge.

It will be understood that the terms “comprise” and “include” and any of their derivatives (e.g. comprises, comprising, includes, including) as used in this specification, and the claims that follow, is to be taken to be inclusive of features to which the term refers, and is not meant to exclude the presence of any additional features unless otherwise stated or implied.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A method for detecting and quantifying elemental mercury vapour in solid samples, the method comprising:
(a) inserting one or more solid sample into an extraction chamber;
(b) introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury vapour, is created and directed to an outlet of the extraction chamber; and
(c) analyzing the loaded gas from the outlet to selectively detect and quantify elemental mercury vapour.

2. The method of claim 1, wherein one or more trap downstream of the outlet receives the loaded gas.

3. The method of claim 2, wherein the one or more trap comprises a first trap that receives the loaded gas and captures the one or more volatile compound, other than elemental mercury vapour, to provide a stripped gas; and wherein an additional trap downstream of the first trap receives the stripped gas and captures the elemental mercury vapor.

4. (canceled)

5. The method of claim 3, wherein the one or more volatile compound captured by the first trap are analysed using gas chromatography or mass spectroscopy analysis to quantify and identify the one or more volatile compound released from the solid sample.

6. The method of claim 3, wherein the elemental mercury vapour captured by the additional trap is analysed by a mercury analyser to quantify the elemental mercury released from the solid sample.

7. The method of claim 2, wherein the one or more trap(s) is/are configured either in series or in parallel.

8. The method of claim 1, wherein the one or more volatile compound comprises volatile organic compounds (VOCs) comprising volatile hydrocarbons.

9. (canceled)

10. The method of claim 8, wherein the volatile hydrocarbons comprise one or more of methane, ethane, propane, n-butane, iso-butane, 1-butene, n-pentane, iso-pentane, hexane, cyclo-pentane, 3-methylpentane, methylcyclopentane, methylcyclohexane, n-heptane, n-octane, furan, methylfuran and BTEX, wherein BTEX comprises any one or more of hydrocarbon compounds benzene, toluene, ethylbenzene and xylene.

11. (canceled)

12. The method of claim 1, wherein the elemental mercury vapour and the one or more volatile compound is released from fluid inclusions in the one or more solid sample.

13. The method of claim 1, wherein the carrier gas comprises either Argon or Nitrogen.

14. The method of claim 1, wherein the one or more solid sample is provided from different locations within a petroleum reservoir, and comprises any one or more of drill cuttings or core samples from the petroleum reservoir.

15. (canceled)

16. The method of claim 1, wherein the conditions include any one or more of crushing, milling, agitating or heating of the one or more solid sample.

17. A system for detecting and quantifying elemental mercury vapour in solid samples, the system comprising:
an extraction chamber comprising a receptacle, an inlet and an outlet in fluid connection with the receptacle, and a comminuting member;
a gas source comprising a carrier gas and in fluid connection with the inlet;
one or more trap in fluid connection with the outlet; and
wherein the extraction chamber is configured to comminute the one or more solid sample to cause the release of one or more volatile compound, including elemental mercury vapour, which is directed toward the outlet by injection of the carrier gas to create a loaded gas, whereby the loaded gas is received by the one or more trap to allow analysis of the loaded gas to selectively detect and quantify elemental mercury vapour.

18. The system of claim 17, wherein the one or more trap comprises a first trap that receives the loaded gas and captures the one or more volatile compound, other than elemental mercury vapour, to provide a stripped gas.

19. The system of claim 18, wherein the one or more trap further comprises an additional trap downstream of the first trap that receives the stripped gas and captures the elemental mercury vapour.

20. The system of claim 19, wherein the elemental mercury vapour captured by the additional trap is analysed to quantify the elemental mercury vapour released from the one or more solid sample and assess the risk of mercury contamination during drilling and production operations of a petroleum reservoir.

21. The system of claim 17, wherein the extraction chamber is agitated to comminute the one or more solid sample via the comminuting member, and the comminuting member is any one of a puck mill, a ball mill, a ring mill, a rock crusher, an anvil or a hammer element.

22. (canceled)

23. The system of claim 21, wherein the comminuted one or more solid sample is weighed and compared to the elemental mercury vapour captured by the additional trap for calculation of fluid inclusion content of the solid sample.

24. (canceled)

25. A method of predicting risk of mercury contamination of a petroleum reservoir by detecting and quantifying elemental mercury vapour in solid samples, the method comprising:
(a) providing one or more solid sample from locations within the petroleum reservoir;
(b) inserting the one or more solid sample into an extraction chamber;
(c) introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury vapour, is created and directed to an outlet of the extraction chamber;
(d) receiving the loaded gas at one or more trap downstream of the outlet to selectively detect elemental mercury vapour within the loaded gas; and
(e) analyzing and quantifying the elemental mercury vapour detected to assess the risk of mercury contamination of the petroleum reservoir.

26. A method of predicting and assessing risk of mercury contamination of a petroleum reservoir, the method comprising:
(a) providing one or more solid sample from a location within the petroleum reservoir;
(b) inserting the one or more solid sample into an extraction chamber;
(c) introducing a carrier gas at an inlet of the extraction chamber and subjecting the one or more solid sample to conditions that cause the release of one or more volatile compound, including elemental mercury vapour, whereby a loaded gas comprising the carrier gas and the one or more volatile compound, including elemental mercury vapour, is created and directed to an outlet of the extraction chamber;
(d) receiving the loaded gas at a first trap downstream of the outlet to capture the one or more volatile compound, other than elemental mercury vapour, to provide a stripped gas;
(e) subsequently receiving the stripped gas at an additional trap downstream of the first trap to capture the elemental mercury vapour, wherein the elemental mercury vapour captured by the additional trap is analysed to quantify the elemental mercury vapour released from the solid sample; and
(f) repeating steps (a) to (e) for one or more subsequent solid sample from alternate locations within the petroleum reservoir, wherein analysed and quantified elemental mercury vapour of the one or more solid sample provided from various locations within the petroleum reservoir are used to predict and assess the risk of mercury contamination of the petroleum reservoir during drilling and production operations.
